(19) United States
(12) Patent Application Publication
(10) Pub. No.: US 2005/0266090 A1
(43) Pub. Date: Dec. 1, 2005

(54) Nanoparticular targeting and therapy

(76) Inventors: Ales Prokop, Nashville, TN (US); Jeffrey M. Davidson, Nashville, TN (US); Gianluca Carlesso, Nashville, TN (US); David Roberts, Bethesda, MD (US)

Correspondence Address: Benjamin Aaron Adler;ADLER & ASSOCIATES, 8011 Candle Lane, Houston, TX, 77071, US

(21) Appl. No.: 11/125,438

(22) Filed: May 10, 2005

Related U.S. Application Data
(63) Continuation-in-part of application No. 10/833,370, filed on Apr. 28, 2004.
(60) Provisional application No. 60/466,375, filed on Apr. 29, 2003.

(51) Int. Cl. A61K 9/14

(52) U.S. Cl. 424/489

(57) ABSTRACT

The present invention provides biocompatible, low molecular weight nanoparticulate formulations that are designed to retain and deliver therapeutics over an extended time course. The therapeutic may be conjugated or adsorbed to the periphery of the corona or conjugated to a core polymer. The nanoparticles comprise targeting ligands also conjugated or adsorbed to the periphery of the corona and/or a contrast agent in the core of the nanoparticle. As such, methods of selective targeting and/or methods of noninvasive imaging using bioluminescence and/or magnetic resonance imaging. Also provided are methods of delivering to and, optionally, imaging of a cell or tissue. Further provided are methods of producing the nanoparticles in batch or continuous mode via simple mixing or laminar flow.

### CROSS-REFERENCE TO RELATED APPLICATIONS

This is a continuation-in-part application of non-provisional U.S. Ser. No. 10/833,370, filed Apr. 28, 2004, which claims benefit of provisional U.S. Ser. No. 60/466,375, filed Apr. 29, 2003, now abandoned.

### FEDERAL FUNDING LEGEND

This invention was produced in part using funds obtained through Grants 5R21HL065982 and 1R0111EB002825-01 from the National Institutes of Health. Consequently, the federal government has certain rights in this invention.

### BACKGROUND OF THE INVENTION

1. Field of the Invention

The present invention relates generally to the fields of sustained drug release and cancer therapy. More specifically, the present invention provides a nanoparticle delivery system capable of targeting tumor vasculature and delivering anti-angiogenic compounds.

2. Description of the Related Art

Development of therapies aimed at inhibiting the growth of new blood vessels is among the most intensively studied approaches in the treatment of cancer (1-2). Since the first mention of tumor vasculature as a potential therapeutic target 30 years ago, understanding of the intricate mechanisms leading to the formation of new blood vessels associated with tumor growth and the spread of metastases has greatly improved (3). This research has led to the discovery of numerous regulatory molecules that influence endothelial cell physiology in vitro and angiogenesis in vivo. They can be divided into two groups: angiogenic factors consisting of growth factors, extracellular matrix molecules and their membrane-bound proteins, e.g. integrins, growth factor receptors, and anti-angiogenic substances.

The anti-angiogenic molecules are believed to have promising applications in the therapy of cancer, arthritis and ocular neovascularization. There are currently more than 30 angiogenesis inhibitors in clinical trials (2), and a multitude of promising new candidates are under investigation in vitro and in animal models. An important therapeutic strategy is the exploitation of endogenous anti-angiogenic molecules to inhibit further tumor growth, to avoid tumor spread and establishment of new distant metastases, or even to shrink the tumor, together with low side effects. Current data demonstrate that tumors and, by inference, capillaries regress when exposed to fragments of plasminogen, i.e., angiostatin, type XVII collagen, i.e., endostatin and peptides derived from thromobospondin-1 (TSP-1) (4-7).

Thrombospondin-1 is a large trimeric glycoprotein composed of three identical 180 kd subunits linked by disulfide bonds. The majority of anti-angiogenic activity is found in the central stalk region of this protein. There are at least two different structural domains within this central stalk region that inhibit neovascularization. Besides TSP-1, there are six other proteins, i.e., fibronectin, laminin, platelet factor4, angiostatin, endostatin and prolactin fragment, in which peptides have been isolated that inhibit angiogenesis. In addition, the dominant negative fragment of Flk1 and analogues of the peptide somatostatin are known to inhibit angiogenesis.

Endostatin is a 20 kDa protein fragment of collagen XVIII. It is a potent inhibitor of tumor angiogenesis and tumor growth (6). Angiostatin is a 38 kDa polypeptide fragment of plasminogen. Whereas plasminogen has no fibrinogenic activity, angiostatin has marked angiogenic activity (4). Angiostatin was isolated when it was observed that the primary tumor suppressed metastases. That is, when the primary tumor was removed, the metastases grew. Administration of angiostatin blocks neo-vascularization and growth of metastases.

The Flk1 receptor is a receptor for vascular endothelial growth factor (VEGF). Flk-1 is expressed exclusively on the surface of the endothelial cells. Once VEGF binds to the receptor, the Flk-1 receptor then homodimerizes to stimulate the endothelial cell to divide. If a mutant receptor of Flk-1 is transfected into the endothelial cells, the mutant receptor dimerizes with the wild-type Flk-1 receptor. In endothelial cells transfected with the mutant Flk-1 receptor, VEGF is unable to stimulate the endothelial cells to divide. Co-administration of a retrovirus carrying the Flk-1 cDNA inhibits tumor growth. This emphasizes that the receptor plays a critical role in the angiogenesis of solid tumors. Chemotherapeutic drugs are often highly toxic and this places a limit on the dose that a patient can tolerate. Peptide-mediated delivery of the drugs selectively to tumor tissue may alleviate this problem, because high concentrations of the drug could be attained within the tumor without affecting normal tissue. Moreover, blood vessels are easily accessible to intravenously administered therapy. Thus, by combining blood vessel destruction with the usual anti-tumor activities of a drug, a drug targeted to the vasculature of tumors can be expected to have increased efficacy and can be used at low enough doses to reduce the toxicity of chemotherapy.

One approach of targeted therapies is based on the specialization of the vasculature of individual organs at the molecular level. Endothelial cells lining blood vessels express tissue-specific markers. Binding of circulating chemotherapeutic agents delivered systemically to endothelial cell surface markers may induce localized cytotoxic effects. Targeting to tumor vasculature is promising as both primary tumor growth and the formation of metastasis depend on the establishment of new blood vessels from preexisting ones. Inhibition of angiogenesis and targeting of the tumor vasculature are highly effective in controlling tumor growth.

Targeting cancer therapy to endothelial cells is a rational approach because a clear correlation exists between proliferation of tumor vessels and tumor growth and malignancy. There are differences of cell membrane structures between tumor endothelial cells and normal endothelial cells which could be used for targeting of vectors. Moreover, tumor endothelial cells are accessible to vector vehicles in spite of the peculiarities of transvascular and interstitial blood flow in tumors. Based on the knowledge of the pharmacokinetics of macromolecules, it can be concluded that targeting tumor endothelial cells should have long blood residence time after intravascular application. A long blood residence time would allow a sufficient attachment to tumor endothelial cells.

Preferential homing of tumor cells and leukocytes to specific organs indicates that tissues carry unique marker molecules accessible to circulating cells. Organ-selective address molecules on endothelial surfaces for lymphocyte homing to various lymphoid organs and to tissues undergoing inflammation have been identified. Endothelial markers responsible for tumor homing to the lungs have also been identified.

A new approach to study organ-selective targeting based on in vivo screening of random peptide sequences has been reported. Peptides capable of mediating selective localization of phage to brain and kidney blood vessels were identified and showed up to 13-fold selectivity for these organs. It is possible to employ such targeting in a therapeutic setting (8-9). One peptide motif contained the sequence Arginine-Glycine-Asparagine embedded in a peptide structure that was shown to bind selectively to aᵥβ₃ and aᵥβ₅ integrins. A second peptide motif that accumulated in tumors contained the sequence Asparagine-Glycine-Arginine, which has been identified as a cell adhesion motif. Other peptides derived from the pathological vasculature have also been identified (10-12).

Based on the principle that tumor growth can be limited by restricting the blood supply, a wide variety of anti-angiogenic strategies have been developed, many of which involve systemic administration of macromolecules as bolus, repeated injections. Although the therapeutic index of some of these treatments may be high, less effort has been focused on sustained or targeted delivery of anti-angiogenic compounds. For example, nanoparticulate delivery systems are particularly suited to delivering a therapeutic, such as a drug, a chemotherapeutic or an immunotherapeutic, to an individual.

The prior art lacks methods of delivering a drug or other therapeutic over an extended time course. Specifically, the prior art is deficient in biocompatible, nanoparticulate formulations that are designed to retain and deliver anti-angiogenic peptides over an extended time course. The present invention fulfills this long-standing need and desire in the art.

### SUMMARY OF THE INVENTION

The present invention is directed to a nanoparticle or a pharmaceutical composition thereof that comprises a water-based core comprising at least one polymer having a low molecular weight and a water-based corona surrounding the core comprising at least one polymer having a low molecular weight of opposite charge to said low molecular weight core polymer(s). The nanoparticle comprises a drug or therapeutic peptide conjugated or adsorbed to a low molecular weight polymer at the periphery of the corona. Alternatively, the drug or therapeutic peptide is conjugated to a low molecular weight polymer comprising the core. The nanoparticle also comprises a targeting ligand or other peptide or polymer or combination thereof conjugated or adsorbed to a low molecular weight polymer at the periphery of the corona.

The present invention also is directed to a method of delivering a drug or therapeutic peptide to a cell or tissue of interest in an individual. The nanoparticles comprising the drug or therapeutic peptide described herein are administered to the individual. The targeting ligand comprising the nanoparticles targets the nanoparticle to the cell or tissue of interest in the individual thereby delivering the drug or therapeutic protein thereto. The present invention is directed to a related method comprising a further step of dispersing the nanoparticles within a hydrophobic matrix to form a thin film upon administration.

The present invention is directed to a related method where the targeting ligand is a glycan. The method comprises the further method step of activating immune cells against the cell or tissue of interest. The present invention is directed to another related method where the nanoparticles comprise a bioluminescent agent or a contrast agent in a polyanionic core. The method comprises a further method step of imaging the cell or tissue to track delivery of the drug or therapeutic agent thereto.

The present invention is directed further to a method of producing a nanoparticle suitable for delivery of a drug or therapeutic protein to a cell or tissue of interest in an individual. The method comprises mixing at least one stream of a solution comprising at least on core polymer of the nanoparticle described herein with at least one stream of a solution comprising at least one corona polymer and the targeting ligand of this nanoparticle. The solution of core polymer(s) or the solution of corona polymer(s) further comprises a drug or therapeutic peptide. Nanoparticles are formed having a complex multipolymeric structure effective to conjugate or adsorb the drug or therapeutic peptide and the targeting ligand thereto such that the complex structure of the nanoparticle is suitable to deliver the drug or therapeutic peptide to the cell or tissue of interest.

The present invention is also directed to related methods of nanoparticle production. A bioluminescent agent or contrast agent is added to the polyanionic core solution. In any of these of methods nanoparticle production, mixing of the streams may utilize simple mixing or laminar flowing. Any of these methods further may comprise washing the nanoparticles and, optionally, cryoprotecting and lyophilizing the nanoparticles.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1 demonstrates that nanoparticle-associated thrombospondin peptide P517 shows enhanced capacity to inhibit endothelial cell growth. Dotted horizontal line indicates FGF-2-mediated cell proliferation (HMVEC cells).

### DETAILED DESCRIPTION OF THE INVENTION

In one embodiment of the present invention there is provided a nanoparticle comprising a water-based core comprising at least one polymer having a low molecular weight; a water-based corona surrounding said core comprising at least one polymer having a low molecular weight of opposite charge to said low molecular weight core polymer(s); a drug or therapeutic peptide conjugated or adsorbed to a low molecular weight polymer at the periphery of the corona or conjugated to a low molecular weight polymer comprising said core; and a targeting ligand or other peptide or polymer or combination thereof conjugated or adsorbed to a low molecular weight polymer at the periphery of the corona.

Further to this embodiment the nanoparticles may comprise a hydrophilic matrix such that a plurality of the nanoparticles described herein are dispersed throughout. In an aspect of these embodiments the corona comprises polycationic polymers and the core comprises polyanionic polymers. In a related aspect the polyanionic core further may comprise a bioluminescent agent, a macromolecular contrast agent or a dynamic contrast enhancing agent.

In all aspects of these embodiments the core and corona low molecular weight polymers may be polyanionic polymers comprising LMW sodium alginate, LMW sodium hyaluronate, pentasodium tripolyphosphate, heparin sulfate, or chondroitin sulfate and polycationic polymers comprising LMW polyvinylamine, spermine hydrochloride, protamine sulfate, poly(methylene-co-guanidine) hydrochloride, polyethyleneimine, polyethyleneimine-ethoxylated, polyethyleneimine-epichlorhydrin modified, quarternized polyamide, or LMW chitosan.

Also in all aspects the drug or therapeutic peptide is conjugated or adsorbed to dextran polyaldehyde, LMW sodium alginate or heparin sulfate in the core. Examples of a drug or therapeutic peptide are a growth factor, a gene or other nucleic acid, angiostatin, endostatin, thrombospondin 1 or a peptide fragment thereof, or thrombospondin 2 or a peptide fragment thereof or a combination thereof. Furthermore in all aspects the targeting ligand may be TSP517, TSP521, apoE, a glycan or other polysaccharide targeted to lectin or lectin targeted to a glycan. Further still in all aspects the other peptide or polymer conjugated or adsorbed to the periphery may be bovine serum albumin, LMW sodium alginate, heparin, methacrylate co-polymer, dextran polyaldehyde, or activated polyethylene glycol.

In one particular aspect the core polymers are LMW sodium alginate and chondroitin sulfate and the corona polymers are spermine hydrochloride and poly(methylene-co-guanidine) hydrochloride. In another particular aspect the core polymers are chondroitin-6-sulfate and heparin sulfate and the corona polymers are spermine hydrochloride and poly(methylene-co-guanidine) hydrochloride. Alternatively, the corona polymer is only spermine hydrochloride. In yet another particular aspect the core polymers are LMW sodium alginate and heparin sulfate and the corona polymers are spermine hydrochloride, and poly(methylene-co-guanidine) hydrochloride. Alternatively, the core polymer is only LMW sodium alginate. In still another particular aspect the core polymer is poly(methylene-co-guanidine) hydrochloride and the corona polymers are chondroitin sulfate and heparin.

In another embodiment of the present invention there is provided a method of delivering a drug or therapeutic peptide to a cell or tissue of interest in an individual, comprising administering the nanoparticles described supra comprising the drug or therapeutic peptide to the individual; and targeting the nanoparticles to the cell or tissue via the targeting ligand comprising the nanoparticles, thereby delivering the drug or therapeutic protein to the cell or tissue in the individual. Further to this embodiment the method comprises dispersing said nanoparticles within a hydrophobic matrix to form a thin film upon administration.

In one aspect of these embodiments the targeting ligand is a glycan where the method further comprises activating immune cells against the cell or tissue of interest. In another aspect the nanoparticles comprise a bioluminescent agent or a contrast agent in a polyanionic core where the method further comprises imaging the cell or tissue to track delivery of the drug or therapeutic agent thereto. In all aspects of these embodiments the cell or tissue of interest may comprise a tumor or tumor vasculature or the tissue may have a wound thereon.

In yet another embodiment of the present invention there is provided a method of producing a nanoparticle suitable for delivery of a drug or therapeutic protein to a cell or tissue of interest in an individual, comprising mixing at least one stream of a solution comprising at least one core polymer of the nanoparticle described supra with at least one stream of a solution comprising at least one corona polymer and the targeting ligand of this nanoparticle where the solution of core polymer(s) or the solution of corona polymer(s) further comprises a drug or therapeutic peptide; and forming nanoparticles having a complex multipolymeric structure effective to conjugate or adsorb the drug or therapeutic peptide and the targeting ligand thereto, such that the complex structure of the nanoparticle is suitable to deliver the drug or therapeutic peptide to the cell or tissue of interest.

Further to this embodiment the method may comprise washing the nanoparticles. Further still the method may comprise cryoprotecting the nanoparticles in a cryopreservation solution and lyophilizing the cryoprotected nanoparticles. In one aspect of these embodiments the mixing step comprises simple mixing of one stream of the core solution and one stream of the corona solution together in a batch mode and stirring the mixed solutions. Alternatively, the mixing step may comprise laminar flowing of one or more streams each of the core solution and of the corona solution together in a continuous mode. In this alternative aspect the laminar flow of at least one of the streams is oscillated. The stream(s) may be oscillated at a frequency of about 5 Hz and 200 Hz. In any of these aspects the solutions may be mixed at a flow ratio of about 1:1 to about 1:12 core polymers:corona polymers.

In one aspect the corona solution comprises polycationic polymers and the core solution comprises polyanionic polymers. Further to this aspect the method comprises adding a bioluminescent agent, a macromolecular contrast agent or a dynamic contrast enhancing agent to the core solution. In all aspects of these embodiments the core polymers individually may be present in a concentration of about 0.01 wt-% to about 0.5 wt-%. The corona polymers individually may be present in a concentration of about 0.01 wt-% to about 1.0 wt-%. The drug or therapeutic peptide may be present in a concentration of about 0.01 wt-% to about 1.0 wt-%. The targeting ligand is present in a concentration about 0.01 wt-% to about 1.0 wt-%.

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof.

As used herein, the term “drug” shall refer to a chemical entity of varying molecular size, both small and large, either naturally occurring or synthetic, exhibiting a therapeutic effect in animals and humans. If not specifically referred to in context, drug may include any therapeutic protein, peptide, antigen or other biomolecules, such as growth factors and genes. A “small” drug may be incorporated within a nanoparticle comprising at least one corona polymer and at least one core polymer of low molecular weight, as defined infra. If not specifically referred to in context “protein” and “peptide” are used interchangeably.

As used herein, the term “microparticulate systems” shall refer to particles having diameter 1-2,000 μm such as microcapsules with a diameter of 100-500 μm or nanoparticles with a diameter range 1-1000 nm with small nanoparticles having a range preferable range of 10-300 nm. Collectively, these systems are denoted as drug delivery vehicles.

As used herein, the term “microcapsule” shall refer to microscopic, i.e., a few micrometers in size to few millimeters, solid object, having an essentially regular spherical shape, exhibiting a polymeric core and a polymeric shell. Usually, the polymeric core and the polymeric shell have opposite charges. For example, a polyanionic core may be covered by a polycationic shell or corona.

As used herein, the term “nanoparticle” shall refer to submicroscopic, i.e., less than 1 micrometer in size, solid object, essentially of regular or semi-regular shape. The particles comprise a polymeric core and a polymeric shell that are opposite in charge. For example, a polyanionic core may be covered by a polycationic shell or corona.

As used herein, the term “polymeric shell” or “corona” refers to the outer layer of the nanoparticle. This layer exerts a partial permeability control. As used herein, the term “polymeric core” shall refer to the inner part of the nanoparticle, usually holding a drug to be delivered. As used herein, the term “polycation” shall refer to a polycationic polymer. As used herein, the term “polyanion” shall refer to a polyanionic polymer. As used herein, the term “low molecular weight” shall refer to a weight less than about 60,000 daltons. As used herein, the term “cryoprotecting” shall refer to substances used for suspension of particles, which upon their water removal in vacuum allow particles to remain in individual and nonaggregating states.

In the description of the present invention, the following abbreviations may be used: BSA—bovine serum albumin; LMW-SA, low molecular weight sodium alginate; LMW-HY, low molecular weight sodium hyaluronate, HS, heparin sulfate; CS, cellulose sulfate; k-carr, kappa carrageenan; LE-PE, low-esterified pectin (polygalacturonic acid); PGA, polyglutamic acid; CMC, carboxymethylcellulose; ChS-6, chondroitin sulfate-6; ChS4, chondroitin sulfate-4; F-68, Pluronic copolymer; PVA, polyvinylamine; LMW-PVA, low molecular weight polyvinylamine 3PP, pentasodium tripolyphosphate; PMCG, poly(methylene-co-guanidine) hydrochloride; SH, spermine hydrochloride; PS, protamine sulfate; PEI, polyethyleneimine; PEI-eth, polyethyleneimine-ethoxylated; PEI-EM, polyethyleneimine, epichlorhydrin modified; Q-PA, quartenized polyamide; pDADMAC-co-acrylamide, polydiallyidimethyl ammonium chloride-co-acrylamide; PBS, phosphate-buffered saline; ECM, extracellular matrix molecule, FGF-2, fibroblast growth factor beta; PDGF, platelet-derived growth factor; P521, P517, peptides derived from TSP-1; TSP-1, trombospondin-1 protein; DPA, dextran polyaldehyde; EDC, 1-ethyl-3-(3-dimethylaminopropyl)carbodiimide hydrochloride; PEG, polyethylene glycol; MPC, methacrylate co-polymer.

The present invention provides a series of biocompatible, nanoparticulate formulations used as drug delivery vehicles that have been designed to retain and deliver drugs or therapeutic peptides over an extended time course. These preparations permit modification to a desirable size, provide adequate mechanical strength and exhibit exceptional permeability and surface characteristics. The present invention particularly provides nanoparticles that confer improved control of the permeability of the particles and the release rate of drug conjugated or adsorbed to the nanoparticle periphery using a targeting ligand and one or more other optional peptides or polymers conjugated with or adsorbed to nanoparticle periphery

Generally, these nanoparticles and may be formed from a variety of materials, including synthetic polymers and biopolymers, e.g., proteins and polysaccharides, and can be used as carriers for drugs and other biotechnology products, such as growth factors and genes or may be used to carry imaging agents. These nanoparticles comprise a polymeric core surrounded by a polymeric shell or corona. corona polymeric matrix into which a drug can be chemically incorporated. Particularly, the drug may be chemically or physically linked or attached to the polymeric corona, for example, via chemical conjugation or physical adsorption.

A multicomponent nanoparticle is formed by polyelectrolyte complexation. In these systems, the multicomponent vehicle, e.g., nanoparticle, may comprise two polymers each in the core and in the corona. Alternatively, one polymer plus two oppositely charged polymers are used to assemble the vehicle or nanoparticle. For example, one polyanion and two polycations or two polyanions and one polycations are used.

Polyanionic polymer components may include HV-sodium alginate, LMW sodium alginate, LMW sodium hyaluronate, heparin sulfate, cellulose sulfate, kappa carrageenan, pentasodium tripolyphosphate, low-esterified pectin (polygalacturonic acid), polyglutamic acid, carboxymethylcellulose, chondroitin sulfate-6, chondroitin sulfate-4, polyvinylamine or LMW polyvinylamine, and collagen. Representative polycationic polymer components include polyvinylamine, spermine hydrochloride, protamine sulfate, polyethyleneimine, polyethyleneimine-ethoxylated, polyethyleneimine, epichlorhydrin modified, quartenized polyamide, polydiallyidimethyl ammonium chloride-co-acrylamide, and chitosan.

Preferably, the nanoparticles comprise one or more polyanionic low molecular weight components, such as, but not limited to, low molecular weight sodium alginate, LMW sodium hyaluronate, pentasodium tripolyphosphate, chondroitin sulfate or heparin sulfate. These LMW polyanionic polymers may form nanoparticles with one or more LMW polycationic polymers, such as, but not limited to, LMW polyvinylamine, spermine hydrochloride, protamine sulfate, poly(methylene-co-guanidine) hydrochloride, polyethyleneimine, polyethyleneimine-ethoxylated, polyethyleneimine-epichlorhydrin modified, quarternized polyamide, or LMW chitosan. More preferably, these low molecular weight nanoparticles comprise a targeting ligand conjugated or adsorbed to a low molecular weight polymer at the periphery of the corona. Most preferably, the drug or therapeutic peptide also is conjugated or adsorbed to a low molecular weight polymer at the coronal periphery, although the drug or therapeutic peptide may be conjugated to a polymer within the nanoparticle core.

Furthermore, the low molecular weight nanoparticles disclosed herein comprising a polycationic corona may include proteins, peptides or other polymers effective to prevent aggregation of the nanoparticles. For example, bovine serum albumin, LMW sodium alginate or heparin may be chemically or physically attached, via conjugation or adsorption, to the polycationic periphery of the nanoparticle.

Although low molecular weight formulations are preferable, nanoparticles comprising one or more high molecular weight or high viscosity polymers may be used to deliver a drug or therapeutic peptide. For example, a high molecular weight nanoparticle may comprise the polyanions high viscosity sodium alginate and cellulose sulfate and the polycations poly(methylene-co-guanidine) hydrochloride (PMCG) and spermine hydrochloride. Such nanoparticles may comprise an inorganic salt, such as calcium chloride, in the polycationic corona. Calcium chloride may be present in a polycationic corona solution for nanoparticle production at a concentration of 0.05 wt-% to 0.2 wt-%. Additionally, the polycationic corona polymers may comprise Pluronic F-68. Pluronic F-68 may comprise a corona solution at a concentration of 0.1 wt-% to 5 wt-%. Furthermore, such nanoparticle may comprise a monovalent or bivalent inorganic salt, such as sodium chloride, calcium chloride, or sodium sulfate, in the polyanionic core. Calcium chloride and sodium chloride may be present individually in a polyanionic core solution for nanoparticle production at a concentration of 0.01 wt-% to 2.0 wt-%. This increases the stability of the nanoparticles and results in, inter alia, increased entrapment efficiency for a more efficacious delivery of a biomolecule, such as a drug or imaging agent, contained within the core of the particle.

A corona of polycationic or polyanionic polymers and a core of polyanionic or polycationic polymers, both formed from low molecular weight polymers, are designed to incorporate a drug or peptide molecule of interest conjugated or adsorbed to a small molecular weight polymer, such as dextran polyaldehyde, LMW sodium alginate or heparin sulfate. Particularly, the drug or peptide may be conjugated or adsorbed to a low molecular weight polymer at the periphery of the corona. The nanoparticles may comprise a therapeutic peptide, protein or drug which is, although not limited to, an anti-angiogenic factor. Representative anti-angiogenic factors include angiostatin, endostatin, thrombospondins 1 and 2 and their fragments, i.e., peptides. The nanoparticles may also comprise growth factors, such as, but not limited to, fibroblast growth factor beta or platelet-derived growth factor.

Alternatively, drugs or therapeutic peptides may be conjugated to a polymer within the nanoparticle core. For example, an anionic antigen may be conjugated to a polyanionic core polymer as an integral part of the multipolymeric complex formed with polycationic corona polymers. A nanoparticle having a polycationic core may incorporate a cationic drug. Non-charged small drugs are conveniently attached to larger molecules, preferably charged polymers.

The corona periphery may be modified further by including a targeting ligand for specific delivery to a cell or tissue site. Preferably, the nanoparticles are targeted to an organ or tissue by a ligand, such as TSP517, TSP521, apoE, polysaccharide capable of targeting to lectin molecule on cell surface or lectin capable of targeting to glycan motif on cell surface. For example, a conjugate of a ligand, for example the peptide TSP-517, with activated PEG may be used to target the nanoparticle to the site of interest. Targeting to tumor vasculature can be mediated by peptide targeting or by glycan or lectin-based ligands attached to the periphery of the nanoparticles. Particularly, the targeting ligand may be conjugated or adsorbed to a low molecular weight polymer comprising the periphery of the corona.

To slow the release rate of the drug carried by the nanoparticles, the drug or peptide molecule can be covalently conjugated through a persistent chemical bond or cross-linked through a dissociable Schiff-base bond with at east one core polymer in the nanoparticle. Physiological reaction conditions are selected that induce a dissociable Schiff-base complex that provides slow drug release. The drug or peptide molecule may include various proteins, growth factors, antigens, or genes in addition to synthetic or naturally occurring chemicals.

In the formation of persistent covalent bond, a water-insoluble drug can be conjugated to a water-soluble polymer to solubilize the drug. Alternatively, one can form a conjugate between a water-soluble polymer and water-soluble drug. The conjugate of drug and polymer is then incorporated into a drug carrier of the present invention, including nanoparticles and microparticles. The entire conjugate of drug and soluble polymer is released from the particles by diffusion or by enzymatic degradation of the delivery vehicle.

Furthermore, the invention includes polymeric complexes in which a gelling polymer and/or a polymer for permeability control which normally are charged polymers of opposite charge to the drug molecules are used to slow the diffusion rate of the charged drugs from the nanoparticles. The gelling polymer is typically a core polymer, such as alginate. The polymer for permeability control is typically a corona (shell) polymer, such as poly(methylene-co-guanidine) hydrochloride or spermine hydrochloride.

In addition to selective targeting of endothelial cells, the nanoparticles also may comprise a noninvasive imaging agent by incorporating a bioluminescence agent, such as luciferase, and/or magnetic resonance imaging constrast agent, such as, a macromolecular contrast agent or dynamic contrast enhanced agent. An example of a contrast agent is, but not limited to, polymeric gadolinium contrast agent. As such, the present invention also provides methods of using the claimed nanoparticles to deliver a drug to a targeted tissue, such as tumor vasculature. When the nanoparticles further incorporate a bioluminescence agent or a contrast agent, simultaneous drug delivery and imaging of the targeted tissue can be performed.

Thus, pharmaceutical compositions may be prepared using a drug encapsulated in the delivery vehicle or linked or attached to the periphery of the delivery vehicle of the present invention. In such a case, the pharmaceutical composition may comprise a drug, e.g., anti-vascularization agent, and a biologically acceptable matrix. Suitable polymeric forms include microcapsules, microparticles, films, polymeric coatings, and nanoparticles.

Prior to use the nanoparticles may be cryoprotected or lyophilized to extend the therapeutic life of the nanoparticle. Cryoprotecting the nanoparticles, with concomitant stabilization, is provided by means of lyophilization. The washed particles are suspended in a cryoprotective solution and lyophilization of the suspension is performed in a suitable lyophilization apparatus. Such cryoprotective solutions may include glycerol, trehalose, sucrose, PEG, PPG, PVP, block polymers of polyoxyethylene and polyoxypropylene, water soluble derivatized celluloses and some other agents at a concentration of 1 wt-% to 10 wt-%.

Because of their small size and suitability for use in injectable formulations. These nanoparticles can be administered locally or systemically. For example, a pharmaceutical composition comprising the nanoparticles of the instant invention may be administered orally, intravenously, nasally, rectally or vaginally, through inhalation to the lung, and by injection into muscle or skin or underneath the skin. Additionally, those polyelectrolyte complexes with a polyanionic or polyanionic/salt core that are administered intravenously demonstrate a greater encapsulation efficiency of the drug and stability in sera. Alternatively, the nanoparticles may be dispersed throughout a biocompatible matrix, such as, a hydrophilic matrix to form a film suitable to cover a wound for delivery of the therapeutic thereto.

A person having ordinary skill in this art would readily be able to determine, without undue experimentation, the appropriate concentrations of the biotechnology products, such as drugs or imagining agents, amounts and routes of administration of the drug delivery vehicle of the present invention to deliver an efficacious dosage of drug or other agent over time. Furthermore, one of ordinary skill in the art may determine treatment regimens and appropriate dosage using the nanoparticles of the present invention without undue experimentation. An appropriate dosage depends on the subject's health, the progression or remission of the disease, the route of administration and the nanoparticle used.

The nanoparticles of the present invention may be prepared by providing a stream of uniformly-sized drops of a charged polymer solution in which the particle size of the drops is submicron or at most only a few microns, collecting these droplets in a stirred reactor provided with a polymeric solution of opposite charge, and reacting the droplets and the solution to form the particles. When the drops of polymer are polyanionic and the receiving polymer solution is cationic, the particles have a polyanionic core and a shell or corona of a polyanionic/polycationic complex. The periphery of the particle has an excess positive charge. Conversely, drops of a stream of cationic solution can be collected in a polyanionic solution. These particles have polycationic core and shell of a polycationic/polyanionic complex with an excess of negative charge on the particle periphery.

Alternatively, the nanoparticles may be prepared utilizing a mixing device, e.g., microfabricated mixing device, of complex geometry, suitable for laminar flowing. Flow rates may be continuous or may be pulsed. The oscillatory flow of at least one fluid provides increased fluid flow for mixing and improved processing. Thus, the process is scaled-up.

Mixing devices that use multiple, reactant fluid streams with very high mixing energy density and enhanced mixing intimacy of reactants provide fast and controlled reaction chemistry not available from conventional batch reaction technology. U.S. Pat. No. 6,221,332 provides a means to develop and manufacture nanomaterials in a process controllable to the “molecular level of mixing. Generally, the microfabricated design, in that the system may be scaled-up, provides a much higher throughput and, unlike batch processes, can be operated continuously.

The mixing device may be coupled to a device, such as an autotitrator, which can measure the size or charge density of nanoparticles, in real time, within the output of the mixing device, providing for feedback and correction of the chemistry of the reacting streams, in terms of ratio of flow of individual streams, pH of the streams, salt content of the streams and, alternatively, ethanol content, as a de-solvating agent, within one of the streams, in order to control the final output of the process

The individual polymers of a polyanionic solution useful in the production process, including crosslinking or conjugating polymers, may have concentrations of 0.01 wt-% to 0.5 wt-%. The drug may be present in the core or corona solutions at a concentration of about 0.01 wt-% to about 1.0 wt-%. The individual components of a polycationic solution may be at a concentration of 0.01 wt-% to 0.5 wt-%. The targeting ligand may be present in the corona solution at a concentration of 0.01 wt-% to 0.5 wt-%.

The following examples are given for the purpose of illustrating various embodiments of the invention and are not meant to limit the present invention in any fashion.

### EXAMPLE 1

Anti-Angiogenic Factor-Loaded Nanoparticle

Particles were generated using a droplet-forming core polyanionic solution of 0.05 wt% LMW sodium alginate (LMW-SA), 0.05 wt% chondroitin sulfate (ChS) and 0.05 wt-% TSP-1 in 0.01 M pH 4.2 acetate buffer, and a corona-forming polycationic solution of 0.05 wt-% spermine hydrochloride (SH) and 0.05 wt-% poly(methylene-co-guanidine) hydrochloride (PMCG). Typical ranges of concentrations for these polymers are 0.03-0.06 wt % for LMW-SA, 0.03-0.06 wt % for ChS, 0.03-0.06 wt % for SH, and 0.035-0.55 wt % for PMCG,.

The polymers were low viscosity sodium alginate (Protanal© LF 5/60) from Drammen, Norway) of average molecular weight 12,000; chondroitin sulfate from Sigma Chemical Co., St. Louis Mo., average molecular weight 20,000; poly(methylene-co-guanidine) hydrochloride (PMCG) from Scientific Polymer Products, Inc. (Ontario, N.Y.), with average molecular weight 5,000; spermine hydrochloride (SH) from Sigma, molecular weight 348.2. TSP-1 (Sigma) is a matricellular anti-angiogenic factor, thrombospondin-1, derived from platelets, average molecular weight 83,000.

The particles were instantly formed and were allowed to react for 1 hour. The nanoparticle size and charge was evaluated in the reaction mixture by centrifugation at 15,000 g. The average size was 230 nm and the average charge 25.2 mV. After two centrifugations and washings with 0.01 M acetate pH 4.2 buffer, the product is colloidally stable at pH 4.2, and in animal sera.

### EXAMPLE 2

Anti-Angiogenic Factor-Loaded Crosslinked Nanoparticle

These particles were generated using the same solutions as in Example 1, except the droplet forming solution contained additional polymer, DPA and ¹²⁵I-labeled TSP-1 instead of nonlabeled TSP-1. DPA is dextran polyaldehyde (CarboMer, Westborough, Mass.) with an average molecular weight of 40,000. The TSP-1 labeling was done by means of a labeling kit (Pierce).

The particles were formed instantaneously, allowed to react for 1-hour and their size and charge evaluated in the reaction mixture. The average size was 250 nm and the average charge 25.5 mV. The particles were separated by washing twice with 0.01 M acetate pH 4.2 buffer and centrifugation and were incubated for 30 min in a HEPES buffer at pH 8.0 to perform the crosslinking reaction between the polymer constituents and TSP-1. Finally, nanoparticles were washed and centrifuged with 0.01 M acetate pH 4.2 buffer.

The DPA/TSP-1 mass ratio was: 0 (no crosslinking), 0.01, 0.05 and 0.1. The higher the ratio of DPA/TSP-1, the slower the release rate of the drug. The Schiff-base product between the anionic groups of TSP and aldehyde group of DPA allowed an adjustment of release via ion exchange. The adjustment is made via the amount of Schiff-base product introduced and the degree of dissociation of this covalent bond, depending on in vitro and in vivo conditions. The release rate was adjusted to any value between 3% and 10% per day, amounting to approximately 30 to 10 days of cumulative delivery time.

The tracer quantity was assayed using a gamma counter and the permeability was assessed via an efflux method (13). Particles with different level of crosslinking have different permeability and drug release rate. More crosslinked nanoparticles would have lower drug release rates. Similar results were obtained when the anionic solution was pre-incubated first at pH 8.0 for 30 minutes and the particles formed after incubation of the solution.

Another set of nanoparticles was made in a similar fashion, except the droplet-forming solution contained different amounts of heparin sulfate (Sigma). The ratios tested were 20:1, 10:1, 2:1, 1:1 and 1:2 of TSP-1:heparin sulfate. Release rates were slowed down to 0.5% to 3% per day in presence of heparin as compared to 50% per day for non-crosslinked nanoparticles. Thus, the drug release rate of the nanoparticles can be adjusted over a wide range to suit different therapeutic needs. The drug release rate can be lowered by increasing the extent of cross-linking or conjugation.

### EXAMPLE 3

Nanoparticles with Covalent Conjugation of Peptide Molecule to the Periphery (Anti-Angiogenic Formulation) and their Targeting In Vivo

A drug peptide or targeting peptide may be conjugated to a 30 polymer to reduce the rate of release of a peptide. TSP-517 is a peptide of 1642 Da derived from the thrombospondin molecule, and has the amino acid sequence KRAKQAGWSHWAA (SEQ ID NO.1). This peptide has a heparin-binding motif and is capable of binding to sites on the tumor vasculature. TSP-517 peptide was synthesized by solid-state chemistry in-house (14).

To incorporate TSP-517 into nanoparticles, the peptide was conjugated to the outer shell of the nanoparticles as in Example 1 in a stoichiometric ratio through a covalent attachment of the ligand directly to the outer surface amino groups of polycations residing on the nanoparticle periphery. 1-ethyl-3-(3-dimethylaminopropyl)carbodiimide hydrochloride (EDC) was used to link acid residues of the targeting ligand with amine-rich regions of the nanoparticle. This method offers mild chemical reaction conditions of incubation at slightly elevated pH and should accentuate further the utility of water-based drug-delivery nanotechnology. This method enabled improved coupling stoichiometry for the expensive peptide ligand. The ligand density is adjusted to a level that minimizes particle aggregation while still promoting specific interactions with model surfaces, e.g., cellular receptors. Specifically, nanoparticles were incubated with 0.03 wt-% EDC in buffer pH 8.5 for 30 min, and washed nanoparticles by centrifugation.

A separate batch of nanoparticles, as in Example 1, was prepared in the presence of a small amount of adenoviral luciferase plasmid in the core polymer solution. The adenoviral construct containing luciferase gene was prepared as follows. 293 adenovirus transformed human embryo kidney cells were grown in Dulbecco's modified Eagle's medium (DMEM) with 10% fetal calf serum (FCS) supplemented with 2 mM L-glutamine. The XbaI/SmaI DNA fragment containing an internal ribosome entry site (IRES) and GFP (Green Fluorescent Protein) isolated from pIRES-GFP (Clontech, Palo Alto, Calif.) and another XbaI/Xhol luciferase DNA fragment cut from pGL-Basic (Promega) were subcloned separately into pShuttle-CMV vector (Quantum Biotechnologies, Montreal, Canada).

The resulting plasmid was co-transformed into BJ5381 cells with pAdEasy-1 adenoviral DNA plasmid that was E1 and E3 deleted and replication-deficient. The recombinant adenoviral construct was linearized with Pac I and transfected into 293 cells in which E1 functions can be complemented in order to produce viral particles. To achieve a large adenovirus preparation, Ad-luc-IRES-GFP was amplified in 293 cells cultured in cell factories (Nalgene Nunc), purified by cesium chloride centrifugation, desalted with PD-10 column (Amersham Pharmacia Biotech, Uppsala, Sweden) and stored at −80° C. The viral titer was determined with the cytopathic effect assay (TCDI₅₀) on 293 cells and calculation was done according to the protocol of Quantum Biotechnologies.

To evaluate biodistribution of targeted nanoparticles, mice that had been implanted with polyvinylalcohol sponges as model wounds representing neovasculature of tumor (15) were administered either free adenoviral luciferase (Ad-luc) plasmid or conjugated TSP-517/PEG nanoparticles containing the same amount of adenovirus by tail vein injection. Luciferase was used for nanoparticle visualization by means of a bioluminiscence CCD camera. Luciferase activity was evaluated 4 days after injection.

Free virus localized predominantly to the liver with minor distribution to lung and spleen and in sponge granulation tissue. In contrast, luciferase expression was more widely distributed in mice injected with TSP-PEG nanoparticles. The lung was a significant reservoir and significant luciferase activity was detected in sponge homogenates. The targeted nanoparticles were much less partitioned into the reticular endothelial system (RES) and more into proliferating endothelial cells and pericytes.

Further optimization of conjugate loading by means of multivalent PEG's can further modify the distribution of nanoparticles in favor of neovascular sites. In addition to TSP517, other targeting peptides such as TSP521, ApoE494 peptide which is a monomeric version of ApoE peptide (16) and Ruoslahti's homing peptide (17) could be used. All these peptides have a capability to bind a corresponding motif on the endothelial cell lining of tumor blood vessels. The method is applicable to plurality of targeting agents, using several different targeting agents.

### EXAMPLE 4

Nanoparticles with Covalent Conjugation of Peptide Molecules to the Nanoparticle Periphery

The attachment of other peptides and proteins, e.g., BSA or FGF-2, is similar to Example 3, yielding different surface densities of attached motifs, depending on peptide concentration. The method is applicable to plurality of targeting agents, using several different targeting agents.

### EXAMPLE 5

Nanoparticles with Peripherally-Adsorbed Peptide

The attachment of the same proteins as in Examples 3 & 4, for example, but not limited to BSA, FGF-2 or P521, is performed via a physical adsorption to the nanoparticle periphery. Convenient pH is used in order to present either cationic or anionic charges or groups for the protein or peptide adsorption to provide oppositely charged groups on the protein/peptide for interaction with the nanoparticle surface. Specifically, in one example, peptide P521was adsorbed from a pH 9 solution by simple 30 min incubation, followed by centrifugation and washing. As the peptide is labeled with FITC fluorescent label using a labeling kit (Sigma), the quantity of adsorbed peptide is evaluated by measuring fluorescence at excitation (nm) and emission (nm) wavelength. The entrapment, that is, adsorption efficiency was estimated to be 40%. The method is applicable to plurality of targeting agents, using several different targeting agents.

### EXAMPLE 6

Testing Biological Activity of Conjugated Nanoparticles In Vitro

For functionalized nanoparticles, both physically, i.e., adsorbed, and covalently attached proteins or ligands, such as FGF-2 or P521, angiogenic (FGF-2) and anti-angiogenic (P521) activity was determined in vitro. For Rhodamine-labeled P521 peptide (Rh-P521) covalently attached to the nanoparticle periphery with an efficiency of attachment of 35%, biological activity (inhibition) was detected as described. Confluent HMVEC cells were starved for 24 h in MCBD 131 (Gibco, Life Technologies) with 1% FBS before splitting the cells into a 96-well plate at density of 10000 cells/well. Cells were cultured for another 24 h in low serum (1% FBS) growth media before treating them with either free Rhodamine-P521 or Rhodamine-P521 coupled to nanoparticles (T-NP) at indicated concentrations in the presence of FGF-2 (10 ng/ml) and heparin (10 lU/ml). As a negative control, cells were also treated with the same amounts of empty nanoparticles (E-NP).

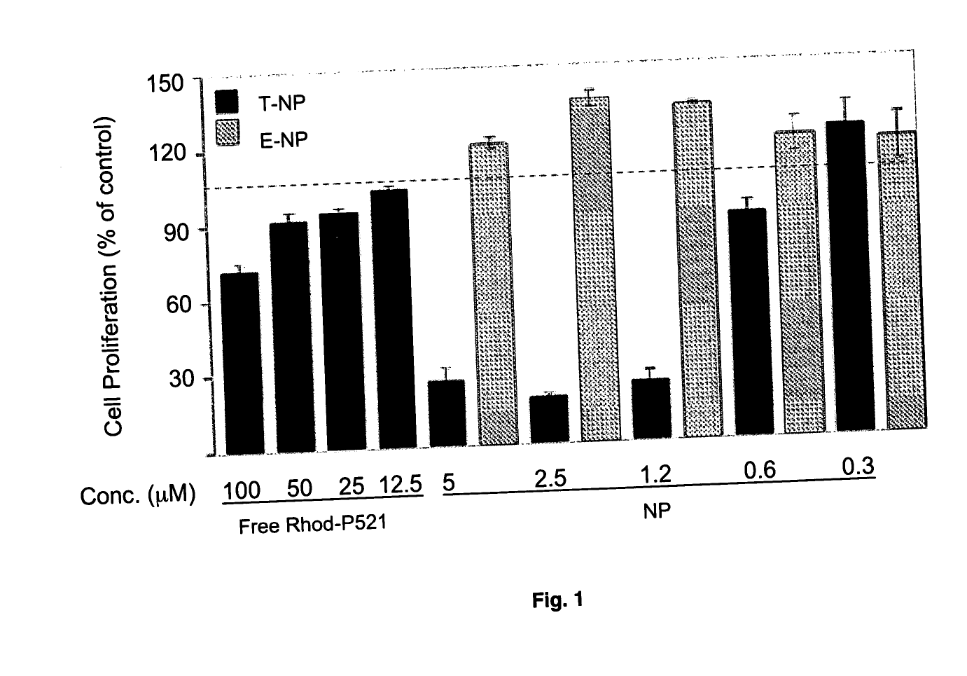

After 72 h culture, a proliferation assay was performed by adding 20 μl of Titer 96 Aqueous One Solution Cell Proliferation Assay (Promega, Madison, Wis.) followed by reading the O.D. at 490 nm. Dotted horizontal line indicates FGF-2-mediated cell proliferation (FIG. 1). The Rh-P521 is much more potent than the free P521 peptide, derived from the Thrombospondin-1 molecule. Other examples included FGF-2 adsorbed at pH 4.2 or covalently attached to the cationically charged NP. In both cases, an activation of HMVEC cell proliferation was detected, in a titrated form (not shown). Thus, these ligands are presented on the surface of nanoparticles in an active form.

In another observation, nanoparticles were prepared with the reverse chemistry featuring negative surface charge, as in Example 8, but different substituent polymers were used with some modification as demonstrated in Example 13. In this case chondroitin sulfate and heparin are the corona polymers and PMCG as the only core polymer. Also, the anti-angiogenic substance was entrapped within the core polymers. In cases of empty nanoparticles, as well as nanoparticles loaded with an anti-angiogenic substance and subsequently crosslinked (Example 2 & 3), when applied in the above in vitro tests, an anti-angiogenic activity was detected. The crosslinking with DPA or EDC was equally effective. Any other crosslinker could be used.

Thus, the crosslinking of the outer shell (corona) polymers generate a biological activity itself, even for non-loaded (anti-angiogenic substance) nanoparticles. Crosslinking is, however, lower, i.e., about 50%, then than for particles decorated/loaded with an anti-angiogenic substance. The nanoparticle crosslinking chemistry itself thus contributes to the biological activity in this case. The mechanism of this activity has not been explained.

### EXAMPLE 7

Preventing the Interaction of Nanoparticles with Serum Proteins

As nanoparticles prepared with the outer periphery cationic charge tend to aggregate in presence of serum, they may cause emboli after their i.v. injection. To prevent their aggregation, BSA or, optionally, another protein/polymer of choice, coating was tested. Nanoparticles prepared as in Example 1 were incubated for 10 min at room temperature in 0.05 wt-% BSA solution in a pH 5 acetate buffer. BSA adsorbed on the particle periphery and surface charge, as measured by Malven instrument, changed to negative values. The colloidal stability was excellent in the presence of serum and in culture media which possess an animal serum. Other negatively charged polymers, at low pH, e.g., 0.05 wt-% alginate or heparin, also could be applied.

### EXAMPLE 8

Preventing Interaction of Nanoparticles with Serum Protein by Processing Means

Nanoparticles were prepared similarly as in Example 1, except that pH of the final re-suspending buffer after two centrifugations and washings was adjusted from 4.2 to 8. Under such conditions, nanoparticles exhibit negative charge. For such nanoparticles, protein serum adsorption is not effective and remains minimal as demonstrated by no shift in the zeta potential. Similarly, nanoparticles were prepared with the same chemistry as in Example 1, except that the two streams were exchanged where the ratio used was 2:16 cation:anion. That means cationic solution was applied as the core solution and anionic solution as the corona solution. Such chemistry is denoted as a reversed chemistry. Again, such nanoparticles, having negative mean charge, did not allow substantial serum protein adsorption at physiologic pH conditions.

### EXAMPLE 9

Preparation of Nanoparticles for Wound Healing Applications

Nanoparticles were prepared as in Example 1, except that a low concentration FGF-2 or PDGF solution (0.01-0.05 wt-%) was used to adsorb or covalently attach to the particle periphery (see Examples 3&4). Likewise, nanoparticles also were prepared as in Example 1, except that 0.01 wt-% of FGF-2 or PDGF were incorporated into the core solution not causing polymer precipitation. Such nanoparticles were tested for angiogenic activity in vitro (see Example 6) and only then incorporated into a 46% Pluronic F-68 solution.

Pluronic behaves like many other thermo-responsive polymers, gelling at elevated temperatures. A rather viscous solution at ambient temperature was used to generate a polymeric film upon contact with an animal body. Specifically, a model full thickness wound was generated in mouse skin by a mechanical punch instrument with an area about 1×1 cm² and nanoparticles were applied onto the wound as an injection in the form of a dispersion within the hydrophilic Pluronic F-68 matrix. This way a sterile closure, i.e., a thin film, was generated in situ due to a fast transformation of dispersion into the gel form at body temperature, 37° C. The dispersed nanoparticulate vehicles provide a sustained release of angiogenic growth factors to potentiate the wound healing. This treatment was repeated every 5 days, allowing for replacement of the medicament as well as wound debriding. Similar results were obtained when the nanoparticles were prepared with growth factor loaded in the core similar to Example 1.

Alternatively, the nanoparticles with angiogenic factors either core-loaded, adsorbed or covalently attached to the periphery of the nanoparticle were applied first as a suspension onto the wound area and subsequently covered with the Pluronic film by injection and in-situ gelling. FGF-2 is fibroblast growth factor beta protein of MW 15,000 and PDGF is platelet-derived growth factor protein of MW 30,000 both from Sigma. Other angiogenic growth factors could also be used.

### EXAMPLE 10

Nanoparticle Stabilization

Nanoparticles were prepared as presented in Example 1, except that the corona solution contained a MPC polymer (NOF, Tokyo, Japan), a methacrylate co-polymer with phosphorylcholine, a highly biocompatible polymer. Because of a cationic moiety of the MPC polymer, this component is involved in he process of nanoparticle assembly together with other interacting polycations. This inclusion within the nanoparticle structure allows for better retention of steric stabilization of the MPC similar to the inclusion of a F-68 Pluronic in corona solution.

LMW polymeric species, providing a charge stabilization is present, do not require F-68 Pluronic for stabilization. Furthermore, in charge stabilized nanoparticles comprising LMW polymers, the F-68 moiety adsorbed to the nanoparticle periphery is diminished to a very level upon the particle three washing steps. MPC is a polymer very similar to cell membrane lipidic components, mimicking their structure. The application of MPC leads to a very stable colloidal structures and allows a decoupling of charge stabilization mechanism from that of steric stabilization by flexible polymers of PEG type (Pluronic). Consequently, such nanoparticles can be prepared at pH different from 4.2 (Example 1), such as at pH 8 and 9, with the zeta potential close to neutrality and still possessing a high colloidal stability.

### EXAMPLE 11

Nanoparticles for Targeting/Activation of Immune System

Nanoparticles were prepared as in Example 3, except the EDC chemistry for covalent attachment was applied for a glucose N-acetyl glucosamine (GIcNAc) polymer, allowing for an activation of the innate immune system. It is contemplated that nanoparticles coated with a specific neoglycan molecules would bind to the immune cell membrane and activate them, starting a whole cascade of events increasing the antitumour activity with involvement of the natural and adaptive immunity. Such nanoparticles would have affinity to lectin-like structures on cancer cells, especially adenocarcinomas, topically delivering a therapeutic payload. GIcNAc-coated nanoparticles are formulated so that their colloidal stability is secured for in vivo applications.

Both entrapment of PAMAM-GIcNAc₈ conjugate and the covalent attachment of the activation motif (GIcNAc₈) are examined. It is contemplated that synthetic multivalent neo-glycoconjugates would provide a new approach to anticancer immune modulation through carbohydrate-mediated immune recognition. Multivalent polyamidoamine dendrimers functionalized with eight N-acetyl-glucosamine residues (PAMAM-GIcNAc₈) were demonstrated to have in vitro high binding affinity for the recombinant lymphocyte receptor rat NKR-P1A (NK1.1 in mice) and to produce in vivo negative modulation of cancer development.

Polyamidoamine (PAMAM) glycodendrimers coated with N-acetyl-glucosamine (PAMAM-GIcNAc₈) were synthesized according to a protocol as previously published (18). Briefly, the synthetic strategy involves reaction of dendritic polyamidoamines (19) with O-acetyl-protected glycosyl isothiocyanates, 2,3,4,6-tetra-O-acetyl-β-N-acetyl-glucosaminyl isothiocyanate for the preparation of PAMAM-GIcNAc₈. Thus, a polyamidoamine dendrimer of generation 1, bearing eight peripheral amino groups lead to O-acetylated isothiourea-bridged glycodendrimers, which give the pure target compounds after deacetylation, using MeONa in MeOH, followed by purification by gel permeation chromatography. PAMAMGIcNAc₈ are obtained as white water-soluble lyophilisates. Purity and structure of the prepared glycodendrimers is evaluated by matrix assisted laser desorption ionization-time of flight (MALDI-TOF) mass spectrometry and NMR analysis (20,21).

Peptide Conjugation to Nanoparticles

The GIcNAc₈ ligand was attached to the outer shell of the nanoparticle in a stoichiometric ratio through a covalent attachment of the ligand directly to the outer surface amino groups of polycations residing on the nanoparticle periphery. EDC is used to link acid residues of the targeting ligand with amine-rich regions of the nanoparticles. This method offers mild chemical reaction conditions, i.e., incubation at slightly elevated pH, for example as in the conditions detailed in Example 3. Likewise, the adsorption was utilized as in the conditions detailed in Example 5.

Testing of Nanoparticles for NK Activation in One In Vitro Model and in One Animal Cancer-Bearing Model and Comparison (as a Control) with PAMAM-GIcNAc8-Activated NK Cells

It also is contemplated that well-defined multivalent neo-glycoconjugates can stimulate an antitumor immune response engaging both innate and acquired immunity. An in vivo cancer model of colorectal carcinoma in rat (AOM) and melanoma in mouse models were utilized. Furthermore, the NK cell interaction with DC cells could be beneficial since the bidirectional interaction of these two cell types is known to enhance NK cell effector function and to induce DC maturation. Thus, GIcNAc-coated nanoparticles could have several functions: NK (NKT, CTL, DC, CD4) cell activation and site-specific targeting of payload to cancer cells.

Specifically, delivery motifs described above was tested via nanoparticles. The nanoparticle's periphery was covalently derivatized, i.e., decorated, with the GIcNAc ligands. The interaction between NK cells and colon carcinoma cells in vitro has been documented, leading to cytotoxic effects. In this context, the interaction was used to enable targeting of nanoparticles to cancer cells. To follow the fate of the compound, a fluorescent marker was conjugated to the tetra-branched semi-component of the dendrimer. Tumor development and immunity were evaluated in C57BL/6 mice. Animals were inoculated with B16F10 melanoma cells and underwent different protocols of PAMAM-GIcNAc₈ administration. Increase of CD69+ cells in the spleen and their appearance inside the tumors, early progressive release of IL-1μ, a later production of INF-γ and IL-2 concomitant to an increment of CD4+ cells was observed. Cytotoxicity assays, performed ex vivo, demonstrated an enhanced NK cell activity proportioned to the percentage of activated NK cells. Other glycomotifs could be used to activate other members of the family of immune cells.

### EXAMPLE 12

Animals Survival Studies

The nanoparticle delivery vehicle similar to that in Example 1 was assembled. It contained core-loaded TSP-1 and corona-conjugated TSP-521 peptide. A slow-release of the core drug peptide is more important for achieving more meaningful therapeutic effects. Thus, to allow for controlled release of the core-loaded peptide, the release rate was adjusted by means of DPA crosslinking. Such crosslinking partially immobilized the corona-entrapped targeting peptide as well as demonstrated in Example 2.

The following three doses of TSP-1 were applied: 150 μg, 80 μg and 10 μg. The cross-linked peptide was designed for slow-delivery over a 10 days period. Moreover, the amount of targeting peptide was adjusted to allow for optimal capture of the nanoparticles in the tumor vasculature. An optimal amount of targeting is that amount allowing for retention but not dislocation of particle within the tumor area. Particles were generated as in Example 1. The nanoparticle periphery was decorated covalently with P521 peptide as prepared in Example 3. The core-loaded TSP-1 functions as a therapeutic anti-angiogenic peptide, whereas the corona associated P521 is a targeting peptide.

For animal studies, a total of 20 tumor bearing mice were used, half of which received an injection of core-loaded TSP-1 nanoparticles with the corona-loaded P521-conjugate. The other half, as controls, received nanoparticles loaded with a corona-attached control scrambled, inactive peptide conjugated to mPEG-SPA. Subcutaneous tumors were produced by local injection of 5×10⁻⁵ 4T1 cells, while liver tumors were produced by injection into the portal vein. Lung metastases occurred spontaneously. In a separate study, tumor response rates were determined for 8 weeks and compared to controls. As a primary measure of the effect of the anti-angiogenic therapy, the animal survival rate was used as the first assessment as shown in Table 1.

TABLE 1
Treatment of Tumors By Targeted Nanoparticles Days (d)

Survival d

| | 14 | 21 | 28 | 35 | 42 | 56 |
|---|---|---|---|---|---|---|
| Test Animals | 100 | 100 | 100 | 90 | 80 | 40 |
| Control Animals | 100 | 100 | 60 | 20 | 0 | 0 |

### EXAMPLE 13

Nanoparticulate Composition with Chondroitin-6-Sulfate and Heparin Sulfate in the Anionic Core

Particles were generated using a droplet-forming polyanionic solution comprising 0.1 wt-% chondroitin-6-sulfate (ChS), 0.1 wt-% heparin sulfate (HS) in water and a corona-forming polycationic solution comprising 0.1 wt-% spermine hydrochloride (SP) and 0.1 wt-% PMCG hydrochloride in pH 4.2 10 mM acetate buffer. Typical range for ChS 0.05-0.15 wt %, for HS 0.05-0.15 wt %, for SP 0.05-0.15 wt % and for PMCG 0.05-0.15 wt %. The anionic solution contained additional polymer, ovalbumin, as a representative protein drug. The amount was about 0.05-4 wt-%. The pH of the polyanionic solution was adjusted within the pH 8.3-11 range by means of diluted sodium hydroxide.

The polymers were low molecular weight chondroitin-6 sulfate (Sigma, St. Louis, Mo.) of average molecular weight 15,000; heparin sulfate, sodium salt (HS) from Sigma (St. Louis, Mo.), with average molecular weight 7,000; poly(methylene-co-guanidine) hydrochloride (PMCG) from Scientific Polymer Products, Inc. (Ontario, N.Y.), with average molecular weight 5,000; and spermine hydrochloride (SH) from Sigma, with molecular weight 348.2.

The particles were instantaneously formed by bringing two polymeric streams, in the ratio 1:8, polyanion/polycation, together in a stirred vessel and allowed to react for 1 hour. The entrapment efficiency was 55% for pH 8.3 of the anionic solution. The entrapment efficiency dramatically increased to 80% when the pH of the anionic solution was increased from pH 8.3 to 11. The nanoparticle size and charge was evaluated in the reaction mixture and after the centrifugation at 15,000 g by means of Malvern instrument (ZetaSizer, Malvern, UK) and by transmission electron microscopy. The average size was 85 nm and the average charge 18.8 mV. The product is stable in 0.9 wt-% saline and in animal sera. Similar results were obtained if only one polycation was used, for example when PMCG was omitted. These nanoparticles can be derivatized for targeting as exemplified in Example 3 and 5, and for slow-release as in Example 2.

### EXAMPLE 14

LMW-Sodium Alginate and Heparin Sulfate in the Anionic Core

Particles were generated using a droplet-forming polyanionic solution comprising 0.05 wt-% low molecular weight sodium alginate (LMW-SA), 0.05 wt-% heparin sulfate (HS) in water and a corona-forming polycationic solution comprising 0.05 wt-% spermine hydrochloride (SH) and 0.05 wt-% PMCG hydrochloride in 10 mM pH 4.2 acetate buffer. A typical range for each of LMW-alginate, HS and SH is about 0.03-0.06 wt-%, for PMCG is about 0.035-0.55 wt-%, and for acetate buffer is about 1-10 mM. The anionic solution contained additional polymer, ovalbumin, as a representative protein drug. The amount was about 0.05-4 wt-%.

The polymers were LMW-alginate (FMC BioPolymers, Philadelphia, Pa.) with an average molecular weight of 12,000; heparin sulfate, sodium salt with an average molecular weight of 7,000, and spermine hydrochloride with an average molecular weight of 348.2, all from Sigma (St Louis, Mo.); and poly(methylene-co-guanidine)hydrochloride with an average molecular weight of 5,000 (Scientific Polymer Products, Inc., Ontario, N.Y.).

The particles were formed instantaneously by bringing two polymeric streams, at a ratio of 1:8 polyanion/polycation, together in a stirred vessel and allowed to react for 1 hour. The entrapment efficiency of ovalbumin was 50%. The nanoparticle size and charge was evaluated in the reaction mixture and after centrifugation at 15,000 g by means of a Malvern instrument (ZetaSizer, Malvern, UK) and by transmission electron microscopy. The average size was about 80 nm and the average charge was 25.2 mV. The product is stable in water, neutral buffers, in 0.9 wt-% saline and in animal sera. Similar results were obtained for nanoparticles formed with only one polyanion, for example, only with LMW-alginate with heparin omitted. These nanoparticles can be derivatized for targeting, as exemplified in Example 3 and 5, and for slow-release, as in Example 2.

### EXAMPLE 15

Preparation of Nanoparticles by Mixing in a Microfabricated Device

Particles may be generated using the chemistry in Examples 13 & 14 with a microfabricated mixing device. The device geometry was similar to that described by Stremler (22) except that it was fitted with two inlets. The size of channels was about 5×5 mm and it was made from Plexiglas (PMMA) polymer. The device allows for laminar mixing in a 3-dimensional channel geometry. The ratio of flow rats was kept 1:8 polyanion/polycation and actual flow rates were 5 and 40 ml/min provided by peristaltic pumps. Once the device reached a steady state over a few minutes, samples were collected and evaluated in terms of optical density (320 nm), size and charge.

Additional runs were made with ovalbumin entrapment, with 0.2-0.5 wt-% of OVA was incorporated into the anionic solution. Entrapment efficiencies were in the range of 50-60%. The microfabricated device provided a much higher throughput rate, 100 mg dry weight/min as compared to 3 mg dry weight/min of batch processing in Example 1. In addition, the microfabricated design allowed for continuous operation.

### EXAMPLE 16

Nanoparticle Preparation Via a Microfabricated Device Plus Fluid Pulsing

Nanoparticles were prepared as in Example 15 except that one or two fluid streams was delivered in a pulsating, i.e., oscillatory, flow regime. For pulsing, a special solenoid valve connected to a frequency power source providing 5-100 Hz frequencies (Precision Dispensing, Bay Village, Ohio) was employed. This set-up allowed independent control of flow rate, as in Example 15, as well as control of the pulsing frequency, i.e., degree of mixing. Mass transfer and mixing is enhanced dramatically with one or two fluids operating in an oscillatory mode (23,24).

The outcome was a more uniform size distribution, evidencing the role of micromixing in the particle assembly process and better process control. With frequencies of 10-30 Hz, the entrapment efficiencies were somewhat higher, as compared to Example 15, when one fluid, the cationic, was oscillated. A useful range of frequencies is between 5 and 200 Hz. Similar results were obtained for both fluids in an oscillatory mode. The oscillatory flow, of at least one fluid, allows for increased fluid flow for mixing and improved processing, as evidenced by dye tracer studies. Thus, higher flow rates were tested, ranging from 10-20 ml/min on the anionic side to 80-160 ml/min on the cationic side. The process scale-up is accomplished.

### EXAMPLE 17

Nanoparticle Cryopreservation and Low-pH and Cryo-Annealing

Nanoparticles were prepared as in Example 1. After production (at pH 4.2), the product was centrifuged twice with washings with pH 4.2 buffer and subjected to lyophilization in 5% trehalose, pH 5.2 solution with a final pH of pH 5.8. The particles were reconstituted in pH 5.8 buffer and the size of particles was measured, i.e., 230 nm diameter and charge 25.2 mV. The size was substantially more acceptable than largely aggregated product re-suspended at pH 5.2 buffer without the cryopreservation. The colloidal stability of the cryopreserved product was followed for up to 5 months without any substantial change.

The following references were cited herein:
1. Deplanque et al., Euro. Jour of Cancer 36:1713-1724 (2000).
2. Hagedorn & Bikfaldi, Critical Reviews in Oncology/Hematology 34:89-110 (2000).
3. Jones & Harris, PPO Update on Principles and Practice of Oncology 14:1-7 (2000).
4. O'Reilly et al., Cell 79:315-328 (1994).
5. O'Reilly et al., Nature Medicine 2:689-692 (1996).
6. O'Reilly et al., Cell 88:277-285 (1997).
7. Streit et al., PNAS USA 96:14888-93 (1999).
8. Steiner, in Angiogenesis: Key Principles-Science, Technology and Medicine, Steiner et al. Eds., Birkhauser, Basel, Switzerland, pp. 449-454 (1992).
9. Pasqualini et al., Cancer Research 60:722-727 (2000).
10. White et al., Hypertension 37 (Pt 2):449-455 (2001).
11. Houston et al., FEBS Letters 492:73-77 (2001).
12. Asai et al., FEBS Letters 510:206-210 (2002).
13. Prokop et al., Advances in Polymer Science 136:52-73 (1998).
14. Bogdanov et al., Neoplasia 1:438-445 (1999).
15. Buckley et al., PNAS USA 82:7340-7344 (1985).
16. Browning et al., J. Exp. Med. 180:1949-1954 (1994).
17. Pasqualini et al., J. Cell Biology 130:5 1189-96 (1995).
18. W B Turnbull, J F Stoddart, Rev Mol Biotechnol 90: 231-255 (2002).
19. Stieger M et al., J Chem Phys 120 (13): 6197-206 (2004).
20. Lindhorst T K and Kieburg C, Angew Chem 108: 2083-2086 (1996).
21. Newkome et al.(eds): Dendrimers and Dendrons. Weinheim, Wiley-VCH, 2001.
22. Liu et al., Jour. of Microelectromechal Systems 9: 190-197 (2000).
23. Nishimura et al., Chemical Engineering Science 48: 1793-1800 (1993).
24. Johnson et al., American Institute of Chemical Engineers Journal 49: 2264-2282 (2003).

Any patents or publications mentioned in this specification are indicative of the levels of those skilled in the art to which the invention pertains. Further, these patents and publications are incorporated by reference herein to the same extent as if each individual publication was specifically and individually incorporated by reference.

One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

1. A nanoparticle comprising:
a water-based core comprising at least one polymer having a low molecular weight;
a water-based corona surrounding said core comprising at least one polymer having a low molecular weight of opposite charge to said low molecular weight core polymer(s);
a drug or therapeutic peptide conjugated or adsorbed to a low molecular weight polymer at the periphery of said corona or conjugated to a low molecular weight polymer comprising said core; and
a targeting ligand or other peptide or polymer or combination thereof conjugated or adsorbed to a low molecular weight polymer at the periphery of said corona.

2. The nanoparticle of claim 1, wherein the corona comprises polycationic polymers and the core comprises polyanionic polymers.

3. The nanoparticle of claim 2, wherein the core further comprises a bioluminescent agent, a macromolecular contrast agent or a dynamic contrast enhancing agent in said polyanionic core.

4. The nanoparticle of claim 1, further comprising a hydrophilic matrix with a plurality of said nanoparticles dispersed throughout.

5. The nanoparticle of claim 1, wherein said low molecular weight polymers are polyanionic polymers comprising LMW sodium alginate, LMW sodium hyaluronate, pentasodium tripolyphosphate, heparin sulfate or chondroitin sulfate and polycationic polymers comprising LMW polyvinylamine, spermine hydrochloride, protamine sulfate, poly(methylene-co-guanidine) hydrochloride, polyethyleneimine, polyethyleneimine-ethoxylated, polyethyleneimine-epichlorhydrin modified, quarternized polyamide, or LMW chitosan.

6. The nanoparticle of claim 1, wherein said core polymers are LMW sodium alginate and chondroitin sulfate and said corona polymers are spermine hydrochloride and poly(methylene-co-guanidine) hydrochloride.

7. The nanoparticle of claim 1, wherein said core polymers are chondroitin-6-sulfate and heparin sulfate and said corona polymers are spermine hydrochloride and poly(methylene-co-guanidine) hydrochloride.

8. The nanoparticle of claim 7, wherein said corona polymer is spermine hydrochloride.

9. The nanoparticle of claim 1, wherein said core polymers are LMW sodium alginate and heparin sulfate and said corona polymers are spermine hydrochloride, and poly(methylene-co-guanidine) hydrochloride.

10. The nanoparticle of claim 9, wherein said core polymer is LMW sodium alginate.

11. The nanoparticle of claim 1, wherein said core polymer is poly(methylene-co-guanidine) hydrochloride and said corona polymers are chondroitin sulfate and heparin.

12. The nanoparticle of claim 1, wherein said drug or therapeutic peptide is conjugated or adsorbed to dextran polyaldehyde, LMW sodium alginate or heparin sulfate.

13. The nanoparticle of claim 1, wherein said drug or therapeutic peptide is a growth factor, a gene or other nucleic acid, angiostatin, endostatin, thrombospondin 1 or a peptide fragment thereof, or thrombospondin 2 or a peptide fragment thereof or a combination thereof.

14. The nanoparticle of claim 1, wherein said targeting ligand is TSP517, TSP521, apoE, a glycan or other polysaccharide targeted to lectin or lectin targeted to a glycan.

15. The nanoparticle of claim 1, wherein said other peptide or polymer conjugated or adsorbed to the periphery is bovine serum albumin, LMW sodium alginate, heparin, methacrylate co-polymer, dextran polyaldehyde or activated polyethylene glycol.

16. A method of delivering a drug or therapeutic peptide to a cell or tissue of interest in an individual, comprising:
administering nanoparticles of claim 1 comprising the drug or therapeutic peptide to said individual; and
targeting said nanoparticles to the cell or tissue via the targeting ligand comprising said nanoparticles, thereby delivering said drug or therapeutic protein to the cell or tissue in the individual.

17. The method of claim 16, further comprising dispersing said nanoparticles within a hydrophobic matrix to form a thin film upon administration.

18. The method of claim 16, wherein said targeting ligand is a glycan, the method further comprising activating immune cells against said cell or tissue of interest.

19. The method of claim 16, wherein said nanoparticles comprise a bioluminescent agent or a contrast agent in a polyanionic core, the method further comprising imaging said cell or tissue to track delivery of said drug or therapeutic agent thereto.

20. The method of claim 16, wherein said cell or tissue of interest comprises a tumor or tumor vasculature or wherein said tissue has a wound thereon.

21. A method of producing a nanoparticle suitable for delivery of a drug or therapeutic protein to a cell or tissue of interest in an individual, comprising:
mixing at least one stream of a solution comprising at least one core polymer of the nanoparticle of claim 1 with at least one stream of a solution comprising at least one corona polymer and the targeting ligand of the nanoparticle of claim 1, said solution of core polymer(s) or said solution of corona polymer(s) further comprising a drug or therapeutic peptide; and
forming nanoparticles having a complex multipolymeric structure effective to conjugate or adsorb said drug or therapeutic peptide and said targeting ligand thereto, wherein the complex structure of said nanoparticle is suitable to deliver the drug or therapeutic peptide to the cell or tissue of interest.

22. The method of claim 21, wherein said corona solution comprises polycationic polymers and said core solution comprises polyanionic polymers.

23. The method of claim 22, further comprising adding a bioluminescent agent, a macromolecular contrast agent or a dynamic contrast enhancing agent to said core solution.

24. The method of claim 21, said mixing step comprising:
simple mixing of one stream of said core solution and one stream of said corona solution together in a batch mode; and
stirring the mixed solutions.

25. The method of claim 21, said mixing step comprising:
laminar flowing of one or more streams each of said core solution and of said corona solution together in a continuous mode.

26. The method of claim 25, wherein the laminar flow of at least one of said streams is oscillated.

27. The method of claim 26, wherein said stream(s) is oscillated at a frequency of about 5 Hz and 200 Hz.

28. The method of claim 21, wherein said solutions are mixed at a flow ratio of about 1:1 to about 1:12 core polymers:corona polymers.

29. The method of claim 21, further comprising:
washing said nanoparticles.

30. The method of claim 29, further comprising:
cryoprotecting said nanoparticles in a cryopreservation solution; and
lyophilizing said cryoprotected nanoparticles.

31. The method of claim 21, wherein said core polymers individually are present in a concentration of about 0.01 wt-% to about 0.5 wt-%.

32. The method of claim 21, wherein said corona polymers individually are present in a concentration of about 0.01 wt-% to about 1.0 wt-%.

33. The method of claim 21, wherein said drug is present in a concentration of about 0.01 wt-% to about 1.0 wt-%.

34. The method of claim 21, wherein said targeting ligand is present in a concentration about 0.01 wt-% to about 1.0 wt-%.


SEQUENCE LISTING (1 sequence)

SEQ ID NO: 1 - protein, 13 aa, artificial sequence
  primer_bind: TSP-517 peptide from thrombospondin
KRAKQAGWSHWAA